(19) United States
(12) Patent Application Publication
(10) Pub. No.: US 2025/0248933 A1
(43) Pub. Date: Aug. 7, 2025

(54) Prophylactic and Therapeutic Agents for Upper Respiratory Tract Infections Caused by Influenza and Other Enveloped Viruses

(71) Applicant: Amy C Brown, Honolulu, HI (US)

(72) Inventor: Amy C Brown, Honolulu, HI (US)

(21) Appl. No.: 19/044,162

(22) Filed: Feb. 3, 2025

Related U.S. Application Data
(63) Continuation of application No. 18/398,070, filed on Dec. 27, 2023, now abandoned.
Application No. 18/398,070 is a continuation of application No. 17/028,276, filed on Sep. 22, 2020, now abandoned.
(60) Provisional application No. 63/006,139, filed on Apr. 7, 2020.
(60) Provisional application No. 62/990,687, filed on Mar. 17, 2020.

(51) Int. Cl. A61K 9/00; A61K 31/19; A61K 31/20; A61K 31/201; A61K 31/23; A61K 31/375; A61K 33/30; A61K 36/185; A61K 36/45; A61K 36/61; A61K 36/73; A61K 36/752; A61K 36/82; A61K 36/87; A61K 36/889; A61K 36/8962; A61K 47/46

(52) U.S. Cl. CPC A61K 9/006; A61K 9/0095; A61K 31/19; A61K 31/20; A61K 31/201; A61K 31/23; A61K 31/375; A61K 33/30; A61K 36/185; A61K 36/45; A61K 36/61; A61K 36/73; A61K 36/752; A61K 36/82; A61K 36/87; A61K 36/889; A61K 36/8962; A61K 47/46; A61K 2236/19

(57) ABSTRACT

The present invention is a novel approach to treating, and even preventing, upper respiratory infections its related symptoms caused by influenza or other lipid-enveloped viruses infiltration and infection in vivo, comprising administering as a gargling agent one or more of clove (plant parts or oil), black tea (with or without caffeine), at least one fatty acid and/or its mono- or di-glyceride form, such as without limitation, coconut water (milk, cream, oil, etc.), caprylic acid, lauric acid (and glycerol monolaurate), monolaurin, myristic acid, oleic acid, palmitoleic acid, or stearic acid, citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonium sidoides and or any extract thereof (African geranium), cranberry or pomegranate juice, zinc, saponified lipid (soap), non-toxic detergent, or vitamin C.

### FIELD OF THE INVENTION

The invention relates to easy treatments and preventives for upper respiratory tract infections and their related symptoms (including, but not limited to sore throat, cough, nasal congestion, and sneezing) caused by influenza and other enveloped viruses, that is, anti-viricidal compositions that are effective against influenza and other enveloped viruses and easy and effective to administer topically to the oral mucosa as a gargle. The gargle may specifically be used to treat diseases caused by lipid enveloped viruses including influenza (Type A, B, C, etc.), COVID-19 (SARS-COV-2), coronavirus caused illnesses, SARS (Severe Acute Respiratory Syndrome), MERS (Middle East Respiratory Syndrome), measles, mumps and Newcastle disease (paramyxoviruses), hemorrhagic diseases such as Ebola (arenaviruses, filoviruses, bunyaviruses, and flaviviruses), and any others.

### BACKGROUND OF THE INVENTION

The common cold and the flu are illnesses that seem very different depending on one's perspective. The sufferer, if not a health care provider him-or herself, will often see the two illnesses as difficult to distinguish. For one thing, symptoms can overlap. Generally, colds are assumed not to include fever and body aches, whereas flu usually does. Moreover, the onslaught of multiple symptoms is an overall experience of feeling awful, and the symptoms of coughing, sneezing, mucous membrane discharge, fever, body aches and so forth tend to blend into one mixed experience—to the point that the patient often does not really care if he or she has a cold versus the flu or other upper and lower respiratory infections caused by enveloped or nonenveloped viruses.

From a scientific and medical point of view. however, the common cold and influenza are radically different health care challenges, despite certain limited similarities. More than 200 different viruses can cause the common cold, but there are really only three virus types that cause the flu—virus types A, B and C. Type B and Type C flu viruses affect (infect) only human beings; Type A flu viruses can infect people and also other animals including ducks, chickens, pigs, whales, horses, and seals, for example. Type C usually causes relatively milder respiratory symptoms in humans, and at this writing there is no vaccine for Influenza virus Type C. Crosstalk, in which Type A flu viruses can infect animal populations and then transition to people, can be particularly devastating—such as Coronaviruses, SARS, and MERS.

Vaccination is without question the first line of influenza defense in the United States at this writing. The relative severity of symptoms and length of illness is so much greater with the flu, in contrast to the common cold, that it is desirable everywhere to prevent the spread of flu wherever possible, and the current thinking is that the best way to do this is with vaccines. Given that there are relatively fewer viruses that are responsible for flu than for the common cold, the vaccination philosophy seems tenable at least at the outset. As a result, “Have You Had Your Flu Shot?” is a common question or placard throughout the United States at this writing, creating a presumption more or less that everyone should have a flu vaccine on a regular basis.

Notwithstanding the first-line flu vaccination phenomenon of present day, no one argues that vaccines have both pluses and minuses. Attempts to quash controversy about side effects from vaccine preservatives are never completely successful. The biophysiological experience of vaccination is never without risks, including death, disease, or at a minimum discomfort and inconvenience. Perhaps worst of all—all influenza vaccines are by definition created from one or more historic influenza viruses, and such a vaccine may well be completely ineffective against the one or more flu viruses that are extant in a given, current disease season. Rationalizations that vaccines designed for “old” viruses still boost the immune system can seem a bit unconvincing. According to the Morbidity and Mortality Weekly Reports (MMWRs), vaccines are not 100% effective against the influenza virus. The Centers of Disease Control and Prevention reported on the “on the basis of data from the U.S. Influenza Vaccine Effectiveness Network on 3,254 children and adults with acute respiratory illness during Nov. 23, 2018-Feb. 2, 2019, that the overall estimated effectiveness of seasonal influenza vaccine for preventing medically attended, laboratory-confirmed influenza virus infection was 47%” [emphasis added, excerpted from Interim Estimates of 2018-19 Seasonal Influenza Vaccine Effectiveness—United States, February 2019 Weekly/Feb. 15, 2019/68(6); 135-139]. A much better approach would be to find not only the antivirals that are viricidal as to influenza Types A, B and C but also to determine the correct route of administration and dosing protocol to render such virucides effective not only for flu treatment, but as a preventive also. With such a preventive and treatment protocol, vaccination could become less of a priority, with concomitant reduction of risk and expense presuming the new virucidal protocol were inexpensive and readily available. A need therefore remains for a virucidal composition and method of administration that effectively prevents or reduces the duration of influenza in a human or animal in need of such prevention or treatment—especially against SARS-COV-2, SARS and MERS.

### SUMMARY OF THE INVENTION

In order to meet this need, the present invention is a protocol of administering—three or more times per day for at least two days—a topical mucosal composition containing an active agent (one or more of) selected from the group consisting of citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonium sidoides and or any extract thereof (African geranium), cranberry or pomegranate juice, zinc, saponified lipid (soap), non-toxic detergent, and vitamin C. The composition is in the form of a gargle, throat spray or lozenge contains one or more active ingredient in said aqueous composition with the following concentrations—citron (20-30% or up to 100% with pH <3), lemon (20-30% or up to 100% with pH <3), lime (20-30% or up to 100% with pH <3), plant acids (citric acid, etc. with amounts necessary to reach <3.0 pH), food grade acids with amounts necessary to reach <3.0 pH) (acetic, lactic, malic, oxalic, benzoic, quinic, tannic, phosphoric and others), vinegar (apple cider, white, balsamic, red wine, wine, etc. with amounts necessary to reach acceptable palatable levels while still being <3.0 pH), garlic (20-30% or up to 80%), gangmei root (at least 13% of each dose), Sambucus nigra (elderberry) (100-400 mg per dose), Pelargonium sidoides and or any extract thereof (African geranium) (1× or the amount found in Umcka) cranberry or pomegranate juice (20-30% or up to 100% with pH <3), zinc (5-40 mg or 10-25 zinc gluconate per dose), saponified lipid (soap) (diluted to acceptable palatability), non-toxic detergent (diluted to acceptable palatability), and vitamin C (1000 mg+/−500 mg per dose). The route of administration is affirmatively gargling—the percussion of the gargling greatly enhances the transport of constituents within an oral mucosal topical composition compared to static topical administration alone. The aliquot for gargling should be approximately 15 ml (½ an ounce or 1 tablespoon). In theory, although applicant does not intend to be bound by the theory, the nosocomial route of transmission of flu viruses inevitably means that the virus will first enter the nasal, perinasal or oral mucosa, so gargling the present admixture becomes a true first line of defense to kill the influenza virus right where it enters the body and begins to inject and replicate. Interestingly, the head-tilted-back action of gargling the present compositions also provides a dissipated particulate treatment throughout the nasal, perinasal and sinus mucosa as well—wiping out the flu virus everywhere it is trying to take initial hold in the body. The present compositions can thus be used in settings where one might be exposed to flu viruses, or can be taken at the first sign of symptoms to kill flu virus throughout the mucosa of the head—before it spreads. Additional diluents and excipients typical in gargle compositions may also be present, with the notable exception of ethanol. Ethanol, known to kill common cold germs in oral mucosa, is ineffective to kill influenza viruses (the present inventor has determined) and its presence in a flu-treatment-only formulation would therefore only provide unwanted mucosal irritation and possible exacerbation of infection of any flu viruses present.

### DETAILED DESCRIPTION OF THE INVENTION

This invention relates to methods and preparations for reducing the duration of upper respiratory infections (URI) and reducing the severity of lipid enveloped virus illnesses starting with sore, scratchy or itchy throat symptoms. More particularly, this invention relates to the remedies comprising medicaments which shorten the duration of URIs, reduce the severity of symptoms, and/or otherwise beneficially treat URIs. These ingredients are believed to be antiviral agents. Such ingredients are combined with a pharmaceutically acceptable carrier suitable for the chosen method and form of administration. The most preferred method of administration is exposure of the oral and oral pharyngeal tissues to the agents for a period of time sufficient for the remedy to be exposed to the oral and oral pharyngeal mucous membranes, such as by gargling. The preferred forms of the remedy for administration therefore include gargles, mouthwashes, throat sprays, and lozenges. In these embodiments, the preparation preferably includes a suitable carrier. and other ingredients such as flavors, stabilizers, lubricants and additional natural or artificial sweeteners.

The “flu” is a time-honored phrase used by both physicians and lay persons alike for the identification of upper respiratory illness caused by viral infection. Influenza is an acute illness in the United States of America, contributing to numerous absences from school and work—as well asworse consequences including but not limited to stay-at-home protocols during epidemics and pandemics. Many if not all of the viruses that cause the flu and other lipid enveloped virus illnesses starting with a sore, scratchy or itchy throat are easily transmitted from host to host. For example, such viruses may be spread in aerosols, so they may be spread through the air by, e.g., coughing or sneezing. Additionally, a number of viruses can survive out of a host's body for extended periods of time, and so can be transmitted by hands and objects.

Since the discovery of rhinovirus in 1956, a considerable body of knowledge has been acquired on the etiology and epidemiology of common colds and influenzas. It is known that the flu is not a single entity, but rather is a group of diseases caused by members of several families of viruses including influenza viruses, parainfluenza viruses, and others. Many over-the-counter remedies for the common cold only treat symptoms. There are over 200 different virus serotypes that can cause the common cold. For example, there are at least 100 serotypes of rhinoviruses alone. It is therefore not possible to build immunity to this many causes of the common cold. There are over 100 coronaviruses, including those causing COVID-19, SARS (Severe Acute Respiratory Syndrome), and MERS (Middle East Respiratory Syndrome). It is also difficult to develop remedies that are effective against such a large number of viruses. Therefore, symptomatic relief has been the traditional recourse.

The aforementioned prescription or over-the-counter products which treat symptoms of the common cold and flu usually contain one or more of the following drugs: antihistamines, decongestants, pain relievers (aspirin, acetaminophen, ibuprofen), cough suppressants, expectorants, and analgesics. These remedies do not reduce the duration of the common cold and flu, are of limited effectiveness in relieving the symptoms of the cold, and are often accompanied by unwelcome side effects. The duration of the average flu varies greatly among individuals. Twenty-five percent of all colds last 14 days, but the average duration of a cold is 7 days, with or without treatment.

Zinc ions have been reported to inhibit the replication of rhinoviruses. See, Korant B D et al., Nature 248:588-590 (1974). Historically, soluble and ionizable zine compounds applied to the oral and oralpharyngeal mucosa have been used to treat common colds and have had some success in shortening the duration of the common cold. See, for example, U.S. Pat. Nos. 5,409,905; 5,286,748; 5,286, 748; RE033465; and U.S. Pat. No. 4,956,385; to Eby Ill. See also U.S. Pat. No. 5,622,724 to Bryce-Smith and U.S. Pat. No. 4,684,528 to Godfrey. Eby claims that after seven days, 86% of 37 zinc-treated subjects were asymptomatic, compared with only 46% of 28 placebo-treated subjects. (Antimicrob. Agents Chemother. 25(1):20-4, 1984). Mossad's double-blind, placebo-controlled study on zinc lozenges revealed that patients treated with zinc lozenges had colds averaging 4.4 days compared to 7.6 days for those on the placebo. (Ann Intern Med 125:81-88, 1996). However, treatment also causes side effects, such as nausea and bad-taste reactions. Mossad. The cold duration thus appears reduced by zinc treatment, but the reduction is not dramatic, especially given the average cold duration of 7 days. The ability of zinc ions to treat lipid coated or lipid-barrier viruses by gargling, specifically, has heretofore been unknown to the knowledge of this inventor. Given the very limited success of current flu remedies, some people say that flu remedies today come no closer to curing the fly than they did thousands of years ago or even since the beginning of recorded medicine. There is no treatment for COVID-19 (SARS-COV-2), SARS (Severe Acute Respiratory Syndrome), MERS (Middle East Respiratory Syndrome). Thus there can be no question as to the need for an improved remedy that will shorten the duration of the flu and other enveloped virus causing infections or even head it off entirely. Flu symptoms are largely the result of the inflammatory response to the viral infection, as described above, rather than the infection itself, so there is some rhyme or reason to the traditional treatments' being primarily symptomatic. Common therapeutic agents not mentioned above include alpha-adrenergic agonist decongestants, antihistamines, analgesics, antipyrrhetics, and antitussives. There is also clinical experience and trials utilizing anticholinergic nasal sprays, mast cell stabilizers, and glucocorticoids. Antibiotics are ineffective except for treating secondary or concurrent bacterial infections.

Botanical Remedies: There is growing interest in complementary medicine. Echinacea is a popular herbal remedy and is thought to reduce cold symptoms due to its ability to stimulate the immune system. Other herbs do have antiviral properties. In an ethnopharmacological screening of medicinal plants used in Yunnan province of China, 16 out of 31 plant extracts (52%) tested positive for antiviral activity, although few plant compounds have been formally tested for activity against rhinovirus. However, there are reports of plant compounds having antiviral activity against other viruses besides rhinovirus, such as Gingyo-osan, Kampo (Japanese herbal), and Tripterygium wilfordii Hook, and elderberry, all of which are reported as effective against the flu virus. Garlic has shown activity against herpes simplex, parainfluenza, vaccinia, vesicular stomatitis, and human rhinovirus type 2. Calanolide A, a coumarin derivative from the tropical rainforest tree Calophyllum lanigerum, is a novel non-nucleoside reverse transcriptase inhibitor for the Human Immunodeficiency Virus. Formulations of Calanolide A have been shown to be safe and well tolerated in healthy HIV-negative humans. Nonetheless, evidence for plant substances effective against influenza viruses are particularly difficult to find, presumably because there are so very many strains of influenza virus.

The present invention's evidence supporting anti-viral mechanisms for the herein disclosed and claimed anti-flu (anti-lipid enveloped virus) ingredients is based on the fact that certain compounds have antiviral action, many originating from botanical sources, and these have been administered, with good empirical results, in the formulation of an antiviral gargle. Numerous plants have evidence of antiviral activity and a few of these are explained in detail below. Many antivirals derived from natural plant sources exist. The question is what works in vitro against flu viruses before or during infection of an animal or human patient? The answer to that question and the crux of this invention is treatment with one or more of citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonitan sidoides and or any extract thereof (African geranium), cranberry or pomegranate juice, zinc, saponified lipid (soap), non-toxic detergent, and vitamin C in a gargle composition with a gargle protocol of at least three times per day for at least two days.

If an antiviral plant substance works in vitro, then it will work as an anti-viral gargle agent in the throat that is actually externally located, even though the nasopharyngeal area appears internal. If a gargle medicament is applied as soon as a person feels their first symptom—a sore, itchy or irritated throat that often signals an oncoming cold or a flu—then there is a strong likelihood of “blocking” or significantly reducing upper respiratory infection symptoms. As part of the prior art. the inventor conducted a double-blind, placebo-controlled clinical trial with 80 subjects (40 per group) showing that her patented gargle remedy reduced the common cold symptoms duration from 7.4 to 4.6 days. The majority of cases (65%) did so in 3 days or less; over one third (39%) of the cold cases were blocked within 2 days; and 15% within the first day.

Based on these results, other case studies, and submitted materials, the inventor received a patent showing that alcohol works well against rhinovirus, but to the inventor's surprise, the same treatment method did not work when she experienced the flu, or in other people who really had the flu and not the common cold as they originally suspected. Why this happened remained a mystery until now. The inventor surmised that the influenza virus was somehow “protected” and did not realize why until finding out serendipitously that some viruses, such as influenza, have a lipid “coat.” Both types of viruses have a protein shell called a capsid that encloses the virus's genetic material. However, some viruses are “enveloped” or “coated” with a lipid envelope. The inventor hypothesized that the non-lipid enveloped viruses such as rhinovirus causing the common cold were susceptible to destruction by alcohol, but lipid enveloped viruses were not. Hence, when she tried her patented method of “blocking” an oncoming flu with her common cold method, it would not work. The flu is far more dangerous with massive global health implications and so the question became, “How best to penetrate or destroy the lipid barrier “blocking” her previous alcohol-based remedy from working?” Her nutrition and food research background made her realize that “soaps” dissolve lipids away in much the same way that dishwashing soap dissipates fat or grease away from a plate. The best way to “dissolve” or destroy the lipid coat surrounding the influenza virus or ANY lipid-enveloped virus would then be something that fundamentally disrupts the lipid coat or barrier—in theory that is, apart from route of administration and whether this would actually work in vivo. The reason that soap works to kill flu virus in vitro is that it is an emulsifier with both hydrophilic and hydrophobic properties that breaks fat into smaller pieces. In fact, the white solid part of common soap bars is sodium stearate, inasmuch as stearic acid is a fatty acid and sodium stearate is its sodium salt. By extension, all of citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonium sidoides and or any extract thereof (African geranium), cranberry juice, zinc, saponified lipid (soap), non-toxic detergent, and vitamin C individually (or in combination) act to break up the lipid barrier of the virus and render the virus inactive.

It is interesting to note—especially with the hindsight of knowing what the present invention is—that there are two mechanisms by which viruses appear to be destroyed or “blocked” by anti-viral substances. During the course of a viral throat infection signaled by a sore, itchy or scratchy throat, there are viruses simultaneously outside and inside of the nasopharyngeal membrane cells. The definition of an infection is that the viruses penetrate the cell membranes to get inside, where replication continues. The growing inflammation of this “invasion” process results in the “itchy, scratchy, and/or sore throat.” The viruses gain access to the throat area through the mouth or nose. If the virus infection location is in the throat, and not the nose, then a gargle medicament would work if the antiviral ingredients are effective against that particular kind of virus—lipid (influenza or ANY lipid-enveloped type virus and resulting type of infection) or non-lipid (rhinovirus types) coating.

Accordingly, the first virus destroying mechanism is a destruction of the viral particles that define the existence of a virus. The viruses could be located inside or outside of the nasopharyngeal membrane cells. antiviral ingredients kill enveloped viruses so they are unable to infect other cells. The second mechanism is the lysis or destruction of cells harboring the virus after it has penetrated the membrane, thereby destroying the host where the virus replicates. Sufficient viral replication (concentration) results in an upper tract respiratory infection and the inventor suggests that symptoms and duration of an upper respiratory tract infection are proportional in part viral concentration, causing disintegration of the plasma membranes of tissue culture cells resulting in cell lysis and death.

Post-completion of the present invention, the inventor believes that both above mechanisms are involved. Destruction of the host cell with the present treatment method destroys the virus' virility for both lipid and no-lipid coated virus. Perhaps the enveloped viruses released from lysed epithelial cells were “protected” from any alcohol treating agent present, if any, and simply went on to infect new cells, whereas alcohol would destroy the protein capsid and ultimately the viral particles inside. In summary, the one or more of citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonium sidoides and or any extract thereof (African geranium), cranberry or pomegranate juice, zinc, saponified lipid (soap), non-toxic detergent, and vitamin C act, according to the invention, to disintegrate the lipid enveloped viruses, of which some are related to upper respiratory infections such as influenza A, B, and C, respiratory syncytial virus (RSV), parainfluenza virus, SARS-COV-2, SARS (Severe Acute Respiratory Syndrome), MERS (Middle East Respiratory Syndrome), measles, mumps and Newcastle disease (paramyxoviruses), hemorrhagic diseases such as Ebola (arenaviruses, filoviruses, bunyaviruses, and flaviviruses), and any other lipid-enveloped viruses. Applying this knowledge, cited studies, and vitro studies collaborated with the inventor through the NIH NAID Respiratory Diseases Branch screening program, along with a few case studies, it is a unique discovery to finally find out why an alcohol gargle does not work against the flu, but that acids and other active agents according to the invention do. This combination of knowledge, with the gargle method that has not been used before for upper respiratory tract infections from lipid enveloped viruses, makes it a unique discovery with prophylactic and therapeutic applications that may very well assist the globe in reducing infection and spread of influenza virus and ALL lipid-enveloped viruses.

Avoiding exposure to the influenza and all viruses is important, because upper respiratory infections can progress to ear infections, and then to lower respiratory tract infections such as croup common in children (3 months to 5 years), bronchitis (infection of air passages between windpipe and lungs) that can continue to pneumonia (infection of the lungs), and then death. “Human respiratory syncytial viruses (RSV-A and RSV-B) are the causative agent in most cases of bronchiolitis, so inhibiting this virus is important. Interestingly, RSV is “surrounded by a lipid bilayer containing the glycoproteins G (receptor binding), F (membrane fusion), and SH (unknown function). Having a simple treatment that is both a preventive and a curative, especially in a global pandemic, is of inestimable value to the modern world.

Given all of the above, it is counterintuitive, yet nonetheless this invention provides methods and compounds for reducing the presence, duration, and severity of a flu and other lipid-enveloped viruses starting with sore, scratchy or itchy throat and other symptoms by the administration of nonalcoholic, antiviral remedy comprising of ingredients that are antiviral when applied topically directly to the mucosa-aqueous solutions of one or more of citron, lemon, lime, plant acids (citric acid, etc.), food grade acids (acetic, lactic, malic, oxalic, benzoic, quinic, tannic, and others and/or their sodium salts), vinegar (apple cider, white, balsamic, red wine, wine, etc.), garlic, gangmei root, Sambucus nigra (elderberry), Pelargonium sidoides and or any extract thereof (African geranium), cranberry juice, zinc, saponified lipid (soap), non-toxic detergent, or vitamin C as discussed above. The preferred route of administration of compositions identified in this paragraph is by gargling. Although once one knows this approach to flu virus eradication and flu disease prevention and treatment it may seem apparent (everything is simple once one already knows it) the truth is that introducing membrane-disrupting active agents against lipid coated viruses works but there have been strong taboos against serious medical prevention and treatment with something so simple as a gargle formulation containing natural ingredients, some of them nutritive! After all, prior treatments for the flu have focused on alleviation of its symptoms. Very few remedies have had any success in reducing the duration of the flu. Further, prior treatments almost always involve vaccines or drugs, the latter which cause side effects, which can be as debilitating as the flu itself. Sometimes the vaccine creates a low-grade episode of flu-like symptoms that dissipate quickly. Heretofore, no one has suggested administering, as a gargle, the listed medicaments—nor established their effectiveness against the influenza virus or other lipid-enveloped viruses.

In a preferred embodiment, the remedy is provided in a form that is not necessarily ingested into the digestive system, but instead facilitates contact between the active agents and the tissues in which the virus is present or through which the virus is likely to enter the body. The remedy can be gargled from a number of forms, including but not limited to a nasal spray, a lozenge, a topical spray, a mouthwash, a traditional gargle formulation, or any form of throat spray. Having said that, the composition is well suited to being swallowed after.

In one embodiment, the medicament is applied with a frequency and/or at a dosage which results in the reduction of the duration of the flu. In another embodiment, the medicament is applied with a frequency and/or at a dosage which results in the reduction of the severity or presence of one or more symptoms of the flu. In yet another embodiment, the medicament is maintained in contact with the mouth and/or throat for a sufficient length of time that reduction of the duration of the flu is achieved. In still another embodiment, the medicament is maintained in contact with the mouth and/or throat fora sufficient length of time that reduction of the severity or presence of one or more symptoms of the flu is achieved. In still another embodiment, the medicament is maintained in contact with the mouth and/or throat for a sufficient length of time that infection by influenza viruses is prevented. Generally, the treatment protocol is twice or three times a day (gargling) for at least two and preferably three consecutive days. More particularly, from the onset of symptoms the first three gargling administrations should ideally occur every two hours, followed by subsequent administration every four to six hours for a total treatment period of two days.

In a related embodiment, the maintenance of contact is achieved through the gargling of a mouthwash for a suitable length of time. In fact, reference to a number of the above routes of administration should be considered as including any mode of administering a gargle aliquot of the medicament. In another related embodiment, the maintenance of contact is achieved by the slow dissolution in the mouth of a suitably sized lozenge and preferably gargling the resultant aqueous composition. In other embodiments, the remedy may be administered in the form of a sublingual or buccal tablet, a syrup, or sublingual liquid drops or pastilles. In yet other embodiments, the remedy is administered in a form that is ingested into the digestive tract, such as tablets, capsules, and liquids.

The remedies of the present invention preferably do not contain any drugs such as antihistamines, decongestants, pain relievers (e.g., aspirin, acetaminophen, ibuprofen), cough suppressants, expectorants, and analgesics. Consequently, the remedies of the present invention also do not produce the side-effects associated with the use of such drugs. Further, the remedies of the present invention are preferably administered in a form which facilitates contact between the active agents and the tissues in which the virus is present. Thus, the remedies of the present invention are preferably not ingested into the digestive system, which is a further reason that the present remedies have not been associated with any side-effects, and the preferred use of all of the compositions disclosed herein is by gargling. Preferably, the gargling protocol accommodates gargling for three accelerated doses on the first day of treatment, that is, once every two hours, followed on that day and at least one following day with additional doses every four to six hours. The dosing on the second day can be, for example, morning, mid-day and evening.

Unlike previous remedies, including zinc lozenges. the remedy of the present invention shows a dramatic blockage of flu symptoms within 12-48 hours of illness. The first 12 to 24 hours of a cold or flu is often unavoidable, as this is the time that patients realize that a cold or flu is actually present or on the way. The remedy of the present invention causes the symptoms to begin to gradually subside as the body's natural immune system is bolstered by the inventive treatment to block the virus invasion. In many cases, a dramatic or complete blockage of the symptoms is effected within 12 to 24 hours of using the remedy of the present invention. The above listed medicaments comprise the remedies of the present invention. It is within the ability of one skilled in the art of herbal therapy or supplementation, pharmacology, and/or clinical medicine to optimize the dosage amount, potency, and frequency of administration in order to accomplish objects such as reducing the duration of a flu, reducing the severity of the symptoms of a flu, and/or preventing infection by an influenza and/or lipid-coated viruses. It is also recognized that these values may differ with such factors as age, weight, and immune status of the subject, severity of illness, and whether the remedy is desired to be used as a treatment for an existing illness or as a prophylaxis.

Other herbal medicaments may optionally be included in the remedies of the present invention. It is recognized that non-drugs which are commonly used to treat cold or flu symptoms, such as drugs such as antihistamines, decongestants, pain relievers (e.g., aspirin, acetaminophen, ibuprofen), cough suppressants, expectorants, and analgesics, may also be included in the remedies of the present invention. However, preferably, the remedies of the present invention do not contain additional drugs besides those listed or benign additional botanicals.

The above described medicaments (nonalcoholic, antiviral ingredients) comprise the remedies of the present invention. It is within the ability of one skilled in the art of pharmacy or food technology to optimize the dosage amount, potency, and frequency of administration in order to accomplish objects such as reducing the duration of a flu, reducing the severity of the symptoms of a flu, and/or preventing infection by a flu virus. It is also recognized that these values may differ with such factors as age, weight, and immune status of the subject, severity of illness, and whether the remedy is desired to be used as a treatment for an existing illness or as a prophylaxis. Also, for the detergent constituent of the invention may be added (in addition to the detergent) or substituted one or more of at least one fatty acid and or its mono- or di-glyceride form, wherein the oral composition contains 0.5-100% of said fatty acid (more specifically, at least 44% lauric acid, 18% myristic, 9% palmitic, 8% caprylic, 6% oleic, 6% capric, and 3% stearic as typically found in coconut oil products). All known fatty acids are embraced by the present invention, as all of them are actively able to disrupt the lipid coat of viruses and any other analogous microbes.

When the remedy is administered as a mouthwash and or gargle formulation, it is most preferable that they gargle with the solution deep in the throat, and then swallow for added protection (however not with the saponified lipids or detergent compositions, these should not be deliberately swallowed and only incidentally ingested in small (harmless) amounts). The person should gargle in such a manner that it reaches the sore, irritated, or scratchy portion of the throat. It is best to gargle as soon as a sore, irritated, or scratchy throat is present (preferably within 12-48 hours). The first three dosages of 15 ml (half ounce or one tablespoon) are taken according to the above schedule. This method and sequence of dosages is extremely important to the success of the treatment. There is no other flu remedy on the market that recommends such a treatment for the flu, however, it is this method that allows this invention to work. It matters not that some of the present medicaments have been known to be antiviral previously—the present teaching of use specifically against lipid barrier influenza and specifically by the route of administration of gargling has been unknown in the art until now. Otherwise, lipid barrier influenza would not still be the problem that it is today! The mouth and throat may be rinsed after application but, if possible, the person undergoing treatment should preferably refrain from rinsing or from drinking any liquids for a period of at least one-half hour or more after gargling. It would also be best not to eat anything for at least a half hour, which eating would also interfere with the contact of the medicaments with the throat area.

Preferably, the remedies do not contain any drugs which are not included in the Pharmacopoeia of the United States, or any herbal ingredients that have been proven over centuries. Preferably, the remedies do not contain any substances or amounts of substances which require evaluation under an NDA (New Drug Application). The term “drug” is not meant to encompass substances which are herbal or are considered to be foods or dietary supplements by the FDA. Additionally, the term “drug” is meant to encompass concentrations of substances for which an NDA would be required by the FDA in larger concentrations. In particular, the remedies of the present invention preferably do not include interferon, interferon inducers, propanediamine, enviroxime, dichloroflaven, 2 {(1,5101-tetrahydro-3H-thiazolo[3,4b]isoquinolin-3-ylidene)amino}-4-thiaz oleacetic acid (S) or sodium polyacrylate.

For the purposes of this disclosure, a subject who is described as “suffering from a flu” is equivalent to a subject who is described as “suffering from the symptoms of a flu,” and both phrases refer to a subject who is experiencing some or all of the above-listed symptoms of the flu and other lipid-enveloped viruses.

The preparations of the invention are suitable for the treatment of an infection by a virus generally recognized as causing, or being associated with, the flu and other lipid-enveloped viruses or the symptoms thereof. As described above, the flu is most commonly caused by influenza viruses (such as influenza A—H3N2, H2n2, H5N1, and influenza B—Victoria or Yamagata). Other diseases caused by lipid enveloped viruses include influenza (Type A, B, C, etc.), COVID-19 (SARS-COV-2), coronavirus caused illnesses, SARS (Severe Acute Respiratory Syndrome), MERS (Middle East Respiratory Syndrome), measles, mumps and Newcastle disease (paramyxoviruses), hemorrhagic diseases such as Ebola (arenaviruses, filoviruses, bunyaviruses, and flaviviruses), and any others. While it is believed that the preparations of the invention are actually virostatic or viricidal, it will be appreciated that this is not known for certain, and it is possible that only symptomatic relief is obtained—and yet reliable symptom relief is indeed obtained within the first 48 hours! Specifically, without wanting to be limited to a specific theory or mechanism of action of the remedies of the present invention, the inventor believes that the remedies function by killing the virus, directly or indirectly with antiviral ingredients, as well as by promoting and enabling the body's own immune system to better destroy, inactivate, and/or prevent the spread of the virus, in part by physically reducing the viral load to be opposed. Likewise, and again without wishing to be so limited by a specific theory, the inventor believes that the remedies prevent infection by destroying and/or inactivating virus with antiviral ingredients before the virus can begin to damage and replicate in tissues and/or by activating promoting and enabling the body's own immune system to better destroy, inactivate, and/or prevent the spread of the virus.

Particularly, the preparations of the invention are suitable for use any time from when the subject first notices any signs of a flu or other disease caused by lipid-enveloped viruses until the symptoms have cleared up. In fact, in some cases, such as for persistent sufferers, or where individually desired, it may be appropriate to continue treatment indefinitely, in the absence of contraindications. Thus, the remedies of the present invention are suitable for acute treatment, chronic treatment, and prophylaxis. Both acute and chronic treatment and prophylaxis will be encompassed by the term “treatment” for purposes of the present disclosure, unless indicated otherwise, either explicitly or from context. The terms “treatment” and “prophylaxis” are used in a broad sense, and extend from symptomatic relief to cure of the infection to general preventative therapy, especially in winter, or for particularly prone individuals. A “subject in need of treatment” includes subjects who have contracted a virus which may cause a flu, subjects who exhibit symptoms of the flu, subjects who are suffering from a flu or from the symptoms of a flu, and those subjects who particularly wish to take preventative measures to avoid infection by a flu virus. Subjects who particularly wish to take preventative measures to avoid infection by a flu virus include those subjects who are or believe they are prone to infection by flu virus and those subjects who are especially vulnerable to suffering severe effects from a flu virus. The especial vulnerability of the latter population of subjects may be due to conditions including old age, young age, or immunocompromisation. In the case of subjects who particularly wish to take preventative measures to avoid infection by a flu virus, the remedies of the present invention may be given as a prophylactic.

When the remedies of the present invention are administered acutely, they are most preferably used beginning at the earliest signs of an oncoming flu or other lipid-enveloped virus disease. For example, a sore throat commonly indicates the onset of a flu or other lipid-enveloped virus disease. The remedy preferably is used according to the above dosing protocols, typically for two days—but the remedies may be used for longer periods, e.g., until the flu or other lipid-enveloped virus disease symptoms are completely gone. To repeat, flu or other lipid-enveloped virus symptoms are typically alleviated or eliminated within two days after commencement of use of the remedy, and so two days is the recommended limit of administration because continued presence of symptoms may indicate a condition other than the flu or lipid-enveloped virus diseases. When the remedies of the present invention are administered prophylactically or chronically, they may be used at lower doses or with lower frequency than is desirable for acute administration. The remedies of the present invention are preferably provided in a form that is not ingested into the digestive system, but instead facilitates contact between the active agents and the tissues in which the virus is present or through which the virus is likely to enter the body. A preferred form other than a mouthwash or gargle is therefore a nasal spray. This form provides topical application of the medicaments to the nasal mucosa. Another form is in the topical application of the medicament in the form of a throat spray that applies to the oral pharyngeal area. A particularly preferred form is a mouthwash. Another particularly preferred form is a lozenge. These particularly preferred forms (mouthwash and lozenge) each provide topical application of the medicaments to the mouth and/or throat (specifically, the oral and oral pharyngeal mucosa). The term “gargling” is to be interpreted broadly and encompasses gargling, swishing, simply holding liquid in the mouth and the back of the throat, and the like. The remedy may also be administered in the form of a sublingual or buccal tablet, a syrup, or sublingual liquid drops or pastilles. These forms also provide for topical application of the medicaments to the mouth and/or throat. The medicament is preferably maintained in contact with the mouth and/or throat for a sufficient length of time that reduction of the duration of the flu is achieved, reduction of the severity or presence of one or more symptoms of the flu is achieved, and/or prevention of infection by flu viruses is achieved. Maintenance of contact may preferably be achieved through the gargling of a mouthwash for a suitable length of time. Maintenance of contact may also preferably be achieved by the slow dissolution in the mouth of a suitably sized lozenge. Maintenance of contact may also be achieved by dissolving a suitable size or amount of, e.g., pastilles, drops, or sub-lingual or buccal tablets in the mouth. The suitable length of contact, and thus the time of gargling, size of lozenge or tablet, or number of pastilles, is readily ascertainable to one of skill in the arts of homeopathy, herbal, and/or clinical medicine.

Further, as with the concentrations of active ingredients to be employed (see above), the length of contact may differ with the characteristics of the subject and the object(s) to be achieved. The remedy may also be administered in a form that is ingested into the digestive tract, such as liquid that is swallowed and coats the mouth and throat, tablets, capsules, and liquids. Methods for formulating nasal sprays, throat sprays, mouthwashes, lozenges, sublingual tablets, buccal tablets, syrups, pastilles, drops, tablets, capsule, and liquids that are suitable carriers for medicaments are well known to the art of pharmaceutical formulation. The formulations may contain a predetermined amount or concentration of at least one medicament according to the present invention. These formulations can be prepared by any suitable pharmaceutical method. The formulations also may vary can vary with the condition and age of the patient and with the object(s) sought to be achieved. The amounts and/or concentrations of medicament to be included may be determined as indicated herein. When formulating a mouthwash or gargle, lozenge, pastilles, or other form which is to be held in the mouth, it may be desirable to include at least one sweetener in the formulation. Examples of suitable sweeteners may be sugars such as fructose, lactose, sucrose, sugar alcohols, and sugar substitutes such as saccharin. Formulations to be held in the mouth may also desirably contain flavoring agents such as, for example, anise, anethole, eucalyptol, wintergreen, licorice, clove, cinnamon, spearmint, cherry, lemon, orange, lime, menthol, peppermint and various combinations thereof. In formulations suitable for nasal administration, the ethanol, herbal, and, optionally, other active ingredients are formulated with a liquid carrier, such as those used in a conventional nasal spray or nasal drops. In formulations suitable for throat administration, the herbal, and, optionally, other active ingredients are formulated with a liquid carrier, such as those used in a conventional throat spray. In a particularly preferred embodiment, the remedy is administered in the form of a mouthwash. Methods for making mouthwashes are well-known in the art. Non-limiting examples of mouthwash compositions, to which the present active agents (at sufficient concentrations) with or without herbal, and, optionally, other active ingredients may be added, are as follows: 0 to 2% by weight of sodium saccharin or other sweetener, or an amount sufficient to provide a sweetening effect equivalent thereto of a sweetening agent; and 0.01 to 1% by weight flavoring agent (such as peppermint oil, spearmint oil, and/or mixtures thereof) 20-95% alcohol (ethanol) q.s. water 5 to 15% v/v sorbitol (or other sugar alcohols) 0.5 to 2.5% w/v surfactant 0.25 to 1% w/v sodium chloride 0.05 to 0.2% w/v insoluble saccharin 0.01 to 0.25% w/v flavoring (such as menthol, thymol, eucalyptol, peppermint oil, and/or mixtures thereof) 0.1 to 2% w/v sodium ricinoleate, acid ingredients 4-6 pH, and 0 to 95% v/v water. When the remedy is administered as a mouthwash or gargle formulation, it is most preferable if the subject does swallow the mouthwash, but also after gargling with the solution deep in the throat. The mouth and throat may be rinsed with the liquid. Further, the subject preferably refrains from drinking any liquids for a period of at least one-half hour or more after gargling. In another particularly preferred embodiment the remedy administered in the form of a lozenge. Formulation of lozenges is well known in the art of pharmaceuticals. As an example, the herbal, and, optionally, other active ingredients may be mixed with a flavored base, usually sucrose and acacia or tragacanth and formulated into lozenges by standard methods. In other embodiments, the remedy may be administered in the form of a sublingual or buccal tablet, a syrup, or sublingual liquid drops or pastilles. Methods of preparing such formulations are well-known in the art of pharmaceuticals. In yet other embodiments, the remedy is administered in a form that is ingested into the digestive tract, such as tablets, capsules, and liquids. For example, the remedy may be administered in the form of gel capsules. It will be recognized that any known means of producing gel capsules can be used in accordance with the present invention. As another example, the remedy may be administered in the form of pressed tablets. Compressed tablets can be prepared by, for example, mixing the herbal, and, optionally, other active ingredients with dry inert ingredients such as carboxymethyl cellulose and compressing or molding in a suitable machine. The tablets optionally can be coated or scored and can be formulated so as to provide slow or controlled release of the active ingredients therein. While the flu remedy ideally omits the inclusion of any ethanol, a cold/flu composite may contain both the disclosed active agents herein as well as 30+% ethanol. The inventor admits that this co-formulation is a compromise, because the ethanol is ineffective against the flu virus and can be undesirably irritating to the oral mucosa, but if the subject or patient does not know whether he or she has the cold or flu or some other lipid-enveloped or non-lipid enveloped virus, the “shotgun” approach of treating both is better than misinterpreting which viral infection has occurred, so as to miss the treatment window of the first 12-48 hours.

Although the invention has been described with particularity above, the invention is only to be limited insofar as set forth in the accompanying claims.

I claim:

1. An oral treatment method to reduce the severity and/or duration of upper respiratory tract infection symptoms (including, but not limited to sore throat, cough, nasal congestion, sneezing, etc.), caused by influenza or other lipid-enveloped viruses resulting in infection comprising: administering to a patient suffering from the upper respiratory tract infections due to influenza, SARS family viruses (Sars-CoV-2, MERS, etc.), or other lipid-enveloped viruses an oral composition in a form suitable for gargling, said oral composition comprising an aqueous composition containing one or more of clove (plant parts or oil), black tea (with or without caffeine), at least one fatty acid, and or its mono- or di-glyceride form, coconut, coconut milk, coconut cream, coconut oil, caprylic acid, lauric acid, glycerol monolaurate, monolaurin, myristic acid, oleic acid, palmitoleic acid, stearic acid, citron, lemon, lime, plant acid, acetic acid, lactic acid, malic acid, oxalic acid, benzoic acid, quinic acid, tannic acid, acid sodium salt, vinegar, apple cider vinegar, white vinegar, balsamic vinegar, red wine vinegar, wine vinegar, garlic, gangmei root, elderberry, Pelargonium sidoides, Pelargonium sidoides extract, African geranium, cranberry juice, pomegranate juice, zinc, saponified lipid (soap), non-toxic detergent, at least one fatty acid and or its mono- or di-glyceride form, wherein 0.5-100% of said fatty acid is present, and vitamin C, in an amount sufficient for the patient to gargle said composition, and further wherein said patient gargles the composition; and repeating the administration of said oral composition at least three times per day or at least every 4-6 hours for at least two days.

2. The oral treatment method according to claim 1 wherein said oral composition is a mouthwash or gargle or throat spray and wherein said oral composition contains one or more ingredients said aqueous composition with the following—citron (20-30% or up to 100% with pH <3), lemon (20-30% or up to 100% with pH <3), lime (20-30% or up to 100% with pH <3), plant acids (citric acid, etc. with amounts necessary to reach <3.0 pH), food grade acids with amounts necessary to reach <3.0 pH) (acetic, lactic, malic, oxalic, benzoic, quinic, tannic, phosphoric and others and/or their sodium salts), vinegar (apple cider, white, balsamic, red wine, wine, etc. with amounts necessary to reach acceptable palatable levels while still being <3.0 pH), garlic (20-30% or up to 80%), gangmei root (at least 13% of each dose), Sambucus nigra (elderberry) (100-400 mg per dose), Pelargonium sidoides and or any extract thereof (African geranium) (1× or the amount found in Umcka) cranberry juice (20-30% or up to 100% with pH <3), zinc (5-40 mg or 10-25 zinc gluconate per dose), saponified lipid (soap) (diluted to acceptable palatability), non-toxic detergent (diluted to acceptable palatability), fatty acid including at least 44% lauric acid, 18% myristic, 9% palmitic, 8% caprylic, 6% oleic, 6% capric, and 3% stearic acids and vitamin C (1000 mg+/−500 mg per dose).

3. The oral treatment method according to claim 1 wherein said oral composition is a nasal spray or swab, with the exception of zinc, and wherein when said fatty acid is present said fatty acid includes at least 44% lauric acid, 18% myristic, 9% palmitic, 8% caprylic, 6% oleic, 6% capric, and 3% stearic acids.

4. The oral treatment method according to claim 1 wherein said oral composition is formulated as a lozenge or troche, with the exception of zinc.

5. The oral treatment method according to claim 1 wherein said oral composition is a beverage to act as a prophylactic agent.

6. The method of claim 1, further comprising the remedy in contact with the mouth, throat or both for a sufficient length of time, with sufficient frequency, or both, that infection by lipid-enveloped viruses is prevented.

7. The oral treatment method according to claim 1 wherein said amount sufficient for said patient to gargle said composition is approximately 15-30 mL.
